(19) United States
(12) Patent Application Publication
(10) Pub. No.: US 2023/0372569 A1
(43) Pub. Date: Nov. 23, 2023

(54) ENVIRONMENTAL DECONTAMINATION

(71) Applicant: Saban Ventures Pty Limited, Macquarie Park, NSW (AU)

(72) Inventor: Vladimir Berentsveig, Sans Souci (AU)

(21) Appl. No.: 18/255,621

(22) PCT Filed: Dec. 7, 2021
(86) PCT No.: PCT/AU2021/051457

§ 371 Date: Jun. 2, 2023

(30) Foreign Application Priority Data
Dec. 7, 2020 (AU) 2020904516

(51) Int. Cl. A61L 9/04; A01N 37/16; A61L 2/26; A61L 2/20; A61L 2/10; A61L 9/12; A61L 9/20

(52) U.S. Cl. CPC A61L 9/044; A01N 37/16; A61L 2/26; A61L 2/20; A61L 2/10; A61L 9/125; A61L 9/20; A61L 2202/11; A61L 2202/15; A61L 2202/25; A61L 2202/13; A61L 2209/12; A61L 2209/135; A61L 2209/134

(57) ABSTRACT

Systems and methods in accordance with embodiments of the invention can operate to decontaminate an environment or surface. In a number of embodiments, an environment or surface is decontaminated by simultaneously exposing it to a combination of peracetic acid vapour and UV-C light. In some embodiments, peracetic acid is aerosolized, then vaporized, and then delivered to a target environment or surface in combination with UV-C to thereby disinfect the target. In various embodiments, the paracetic acid is aerosolized using either a mesh nebulizer or a pneumatic nebulizer.

### FIELD OF THE INVENTION

The present invention relates to methods and apparatus for environmental decontamination, in particular for the decontamination of hospitals, clinics, workspaces and so on that may be infected with bacteria, fungi, viruses, or fungal or bacterial spores.

The invention has been developed primarily for use in a healthcare environment but it will be appreciated by those skilled in the art that it is equally applicable to homes, offices, schools, laboratories, factories and other public spaces where pathogens can be transferred by contact with surfaces or objects found within the environment.

### BACKGROUND OF THE INVENTION

Any discussion of the prior art throughout the specification should in no way be considered as an admission that such prior art is widely known or forms part of common general knowledge in the field.

Considerable attention has been devoted to the disinfection of medical instruments as a means of preventing transmission of pathogens from one patient to another. Specialist medical instruments are generally reprocessed after use to reduce or eliminate pathogens or bioburden that can harbour such pathogens. Other medical instruments and supplies are manufactured for a single use, after which they are disposed of. However, large numbers of hospital patients acquire potentially serious infections in hospital. These hospital acquired (nosocomial) infections do not arise directly as a result of medical procedures but rather arise as a result of environmental contamination or staff-to-patient or patient-to-patient contamination.

The health-care facility environment is commonly implicated in disease transmission and not only among patients who are immunocompromised. Inadvertent exposures to environmental pathogens (e.g., Aspergillus spp. and Legionella spp.) or airborne pathogens (e.g., Mycobacterium tuberculosis and varicella-zoster virus) can result in adverse patient outcomes and cause illness among health-care workers. The number of hospital-acquired infections (HAIs) has been growing exponentially worldwide since 1980, especially due to the emergence and wide spread of multidrug-resistant (MDR) bacteria. Multidrug resistance is an intrinsic and inevitable aspect of microbial survival and has been a major problem in the treatment of bacterial infections.

It has been demonstrated that the increased incidence of HAIs is related to cross-infections from patient to patient or hospital staff to patient and to the presence of pathogenic microorganisms that are selected and maintained within the hospital environment (including equipment).

Pathogens can spread from patient to patient through contact with inanimate surfaces, including medical equipment and the immediate patient environment. There is clinical evidence suggesting an association between poor environmental hygiene and the transmission of microorganisms causing HAIs.

The potential for contaminated environmental surfaces to contribute to pathogen transmission depends on two important factors: the pathogens must survive on dry surfaces and the contamination has to occur on surfaces commonly touched by patients and healthcare staff at a sufficiently high level to enable transmission to patients.

The most frequently contaminated surfaces are floors, doorknobs, television remote control devices, bed-frame, lockers, mattresses, bedside tables and toilet seats in rooms previously occupied by an infected patient. It was found that ˜50% of commodes, toilet floors and bed frames sampled at a hospital were contaminated with C. difficile.

One-third of nosocomial infections are considered preventable. The Centers for Disease Control and Prevention (CDC) in the US estimates 2 million people in the United States are infected annually by hospital-acquired infections, resulting in around 20,000 deaths. The most common nosocomial infections are of the urinary tract, surgical site and various pneumonias.

The likelihood of contracting a hospital acquired infection increases in proportion to the microorganism load in the environment. A reduction in microbial bad produces a concomitant reduction in the likelihood of infection. Any action that can reduce the environmental microorganism load would be expected to have the effect of improved outcome in the patient population. It is not necessary to achieve high level disinfection (HLD, a 6 log reduction in microorganisms) in the environment in order for environmental disinfection to provide significant benefits. A modest reduction in environmental bacteria (for example between a 5 log reduction in microorganisms and a 2 log reduction in microorganisms) will lead to observable reductions in hospital acquired infections. The greater the reduction in microbial load, the more beneficial the effect on preventing environmentally acquired infections.

The most commonly used method for disinfecting such large spaces and surfaces involves the use of reactive gases such as ozone, chlorine dioxide or ethylene oxide which are corrosive and toxic, or sprays of aldehydes such as glutaraldehyde or formaldehyde, which are extremely toxic and which leave potentially harmful residues on surfaces. Steam is sometimes used but this is user intensive and potentially hazardous to the operator because of the high temperatures involved. Steam is not useful in every case as it can damage delicate materials and it leaves a dense, moist film on the surface which may lead to rusting.

### SUMMARY OF THE INVENTION

From a health and environmental perspective it would be preferable to use hydrogen peroxide or peracetic acid as a disinfectant. Nebulized or vaporized peracetic acid or hydrogen peroxide are known disinfectants. They are effective at low concentration.

In order to provide surfaces which are residue free, hydrogen peroxide or peracetic acid need to be provided in very low quantities. Residues are undesirable as they provide a cold damp feel to the touch, and can be harmful to the skin and eyes of those present in the room. Obtaining optimal disinfection for the amount of peroxy sterilant used is very important.

It is an object of this invention to provide a method for disinfecting a large area or disinfecting a volume and which avoids or ameliorates at least some of the disadvantages of the prior art, or provide a useful alternative. It is a further object of the invention to provide improved apparatus and improved fumigants for carrying out the method, or provide a useful alternative.

Accordingly, many embodiments of the invention provide a method of decontaminating an environment or surface comprising the step of simultaneously subjecting said environment or surface to a combination of peracetic acid vapour and UV-C light.

The peracetic acid vapour and UV-C light can provide a synergistic reduction in the amount of microorganisms (e.g. fungi, viruses and most preferably bacteria) present.

The environment may be a closed environment, such as room. Preferably the environment is closed such that the changed volume of the environment does not exceed more than five air changes per hour, more preferably three air changes per hour and even more preferably, the changed volume of the environment does not exceed one air change per hour.

An “air change per hour” is a measure of the air volume added to or removed from a space divided by the volume of the space. Air changes per hour is a measure of how many times the air within a defined space is replaced.

The peracetic acid vapour may be generated from a 2% w/w-15% w/w aqueous peracetic acid solution, for example, a 2% w/w-10% w/w aqueous peracetic acid solution, a 2% w/w-7% w/w aqueous peracetic acid solution or a 5% w/w aqueous peracetic acid solution. However, any suitable concentration can be implemented. For example, ˜35% w/w aqueous peracetic acid solutions are commercially available.

The UV-C light has a wavelength of 100-280 nm, preferably 200-280 nm (e.g. 220 nm to 270 nm, 240 nm to 260 nm, about 250 nm), more preferably about 254 nm.

In one embodiment, the peracetic acid vapour and UV-C light emanate from a single locus. Alternatively, the peracetic acid vapour and UV-C light may emanate from multiple loci, which may be spaced around the environment (e.g. 1 m or more apart from each other in any direction).

In one embodiment, when a room is being decontaminated, the peracetic acid is directed outwardly from a locus across the room. The UV-C may also be directed outwardly from a locus across the room. The UV-C locus may be the same as or different from the peracetic acid locus.

In one embodiment, when a room is being decontaminated, the peracetic acid is directed outwardly from one or more loci across the room. The UV-C may also be directed outwardly from one or more loci across the room. The UV-C loci may be the same as one or more of the peracetic acid loci or different from one or more of the peracetic acid loci.

The peracetic acid vapour is preferably prepared by nebulization of a peracetic acid solution to an aerosol and subsequent evaporation of said aerosol to form peracetic acid vapour. Preferably the aerosol droplets are relatively uniform and small, such that a majority of the droplets (by number) are of a size of 4 μm or less.

Preferably more than 50% of the droplets (by number) (e.g. 55% or more, e.g. 60% or more) are of a size of 5 μm or less (e.g. 4 μm or less). Preferably the median droplet size is between 2 μm and 10 μm, e.g. between 4 μm and 5 μm. Preferably 90% or more (e.g. 95% or more, e.g. 99% or more) of the droplets (by number) are of a size between 2 μm and 50 μm. In one embodiment at least 90%, e.g. at least 95%, e.g. at least 99%, of the droplets of a size of 25 micrometers or less. In one embodiment at least 90%, e.g. at least 95%, e.g. at least 99%, of the droplets of a size of 10 micrometers or less.

For a constant injection liquid volume, reducing the droplet diameter increases the surface area and therefore the rate of evaporation. A narrow droplet size distribution may be preferable.

There are a variety of different machines, which are suitable to measure particle size distribution (e.g. Malvern Mastersizer S (Malvern Instruments Ltd, Worcestershire, UK). All these machines using laser diffraction (light scattering effect) to characterize particle size distribution.

Any form of nebulization may be used. For example, nebulization may be by way of an ultrasonic nebulizer (e.g. operating at 2.4 MHz) but it can also be by way of a pneumatic nebulizer. In addition, nebulization may be by way of a mesh nebulizer.

In some embodiments of the invention, what is provided is a device for projecting peracetic acid vapour, the device comprising a nebulizer to produce a mist of peracetic acid droplets and a mechanism to evaporate the peracetic acid droplets to peracetic acid vapour and to propel the vapour away from the device. The nebulizer may be, for example, a pneumatic nebulizer. The mechanism may be, for example, a fan or compressed air.

In some embodiments of the invention, what is provided is a device for projecting peracetic acid vapour, the device comprising a nebulizer to produce a mist of peracetic acid droplets and a fan to evaporate the peracetic acid droplets to peracetic acid vapour and to propel the vapour away from the device. The nebulizer may be, for example, a pneumatic nebulizer.

In some embodiments of the invention, what is provided is a device for projecting peracetic acid vapour, the device comprising a nebulizer to produce a mist of peracetic acid droplets, a first mechanism to propel the droplets to a location adjacent the device and a second mechanism to evaporate the peracetic acid droplets to peracetic acid vapour and to propel the vapour away from the device. In some embodiments, the first mechanism to propel the droplets to a location adjacent the device is a fan. In some embodiments, the second mechanism to evaporate the peracetic acid droplets to peracetic acid vapour and to propel the vapour away from the device is a fan. In some embodiments, the first mechanism to propel the droplets to a location adjacent the device comprises compressed air. In some embodiments, the second mechanism to evaporate the peracetic acid droplets to peracetic acid vapour and to propel the vapour away from the device comprises compressed air. The nebulizer may be, for example, an ultrasonic nebulizer, a mesh nebulizer or a pneumatic nebulizer.

In some embodiments of the invention, what is provided is a device for projecting peracetic acid vapour, the device comprising a nebulizer to produce a mist of peracetic acid droplets, a first fan to propel the droplets to a location adjacent the device and a second fan to evaporate the peracetic acid droplets to peracetic acid vapour and to propel the vapour away from the device. The nebulizer may be, for example, an ultrasonic nebulizer, a mesh nebulizer or a pneumatic nebulizer.

While fans are mentioned, it should of course be appreciated that any mechanism to propel the droplets to a location adjacent the device can be implemented in accordance with embodiments of the invention. For example, in some embodiments, the mechanism may comprise compressed air.

While fans are mentioned, it should of course be appreciated that any mechanism to evaporate the peracetic acid droplets to peracetic acid vapour and to propel the vapour away from the device can be implemented in accordance with embodiments of the invention. For example, in some embodiments, the mechanism may comprise compressed air.

Preferably the peracetic acid is propelled away from the device in a predetermined direction. This may be accomplished by a fin, blade, louver or jet, intermediate the mist and the second mechanism, to provide directional flow of the aerosol vapour. In one embodiment, the second mechanism can be a fan (e.g. a second fan).

Preferably, the device further comprises a UV-C source. The UV-C source may project UV-C radiation in a predetermined direction, most preferably in the same direction as the aerosol vapour is propelled.

Preferably, the device further comprises a humidifier.

### Definitions

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

The UV-C light has a wavelength of 100-280 nm, preferably 200-280 nm (e.g. 220 nm to 270 nm, 240 nm to 260 nm, about 250 nm), more preferably about 254 nm. The term “light” and “UV-C light”, as used in the specification, refer to UV-C electromagnetic radiation.

Droplet size refers to the size of a droplet in its largest dimension. Thus a spherical droplet of size 4 μm is understood to have a diameter of 4 μm.

The terms “mist” and “aerosol” are used interchangeably to refer to a suspension of particles dispersed in air or gas. Mist refers to liquid droplets even though they are very small.

“Vapour” refers to separate molecules or small dusters dispersed in air. The term “vapour” is different from mist and aerosol. As noted elsewhere in the specification, evaporation of the peracetic acid aerosol (or mist) forms peracetic acid vapour. Mist can be detected using Tyndall effect (light scattering effect), but vapour does not scatter light and is invisible in a laser beam.

“Aerosol vapour” refers to vapour generated by evaporation of an aerosol.

Peracetic acid is sold as an equilibrium mixture of peracetic acid, hydrogen peroxide, acetic acid and water:

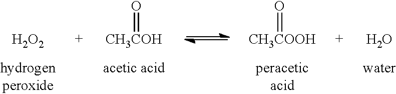

Peracetic acid mist/aerosol refers to a mist/aerosol which comprises an equilibrium mixture of peracetic acid, hydrogen peroxide, acetic acid and water.

Peracetic acid droplets refers to droplets which comprise an equilibrium mixture of peracetic acid, hydrogen peroxide, acetic acid and water.

Peracetic acid vapour refers to a vapour which comprises an equilibrium mixture of peracetic acid, hydrogen peroxide, acetic acid and water.

Abbreviations:
PAA Peracetic acid
H₂O₂ Hydrogen peroxide
AC Activated carbon
ACH Activated carbon honeycomb
PZT Pb(ZrTi), Lead Zirconate Titanate

### BRIEF DESCRIPTION OF THE DRAWINGS

Preferred embodiments of the invention will now be described, by way of example only, with reference to the accompanying drawings in which:

FIG. 1 shows the droplet size distribution of the mist produced by a 2.4 MHz ultrasonic nebulizer.

FIG. 2 shows the flow of peroxide vapour in an embodiment of the present invention.

FIG. 3a shows an embodiment of a device for the preparation and dispensation of peracetic acid vapour using an ultrasonic nebulizer.

FIG. 3b shows an embodiment of a device for the preparation and dispensation of peracetic acid vapour using a mesh nebulizer.

FIG. 3c shows an embodiment of a device for the preparation and dispensation of peracetic acid vapour using a pneumatic nebulizer.

FIG. 4 shows the UV-C light irradiating a room.

FIG. 5 shows a surface plot of the disinfective ability of peracetic acid.

FIG. 6 shows a surface plot of the disinfective ability of UV-C.

FIG. 7 shows a surface plot of the disinfective ability of UV-C and peracetic acid in combination.

FIGS. 8a and 8b show the positions of test dishes used to determine efficacy of an embodiment of the invention.

FIG. 9 shows the synergistic effect of UV-C and peracetic acid according to the present invention.

FIG. 10 shows some commercially available Activated Carbon Honeycombs.

FIG. 11 shows tests of different PAA remediation options.

FIG. 12 shows removal of PAA and H₂O₂ by Honeycomb Catalytic Cartridge.

FIG. 13a shows an example of a mesh nebulizer.

FIG. 13b shows an embodiment of a device for the preparation and dispensation of peracetic acid aerosol using a mesh nebulizer.

FIG. 13c shows an example of a pneumatic nebulizer.

FIG. 14 shows a prototype of a device comprising a nebulizer, UVC lamps, a humidifier and a catalytic cartridge.

FIG. 15a shows bio-decontamination efficiency using a combinatorial approach of PAA and UVC at different humidity levels in the room.

FIG. 15b shows the target positions 1 to 6 in the room as referred to on the x-axis of FIG. 20.

FIG. 16 shows the log 10 reduction with UVC only, with PAA only and with UVC in combination with PAA.

FIG. 17 shows the kinetics of Staphylococcus aureus deactivation with PAA, with and without UVC.

FIG. 18 shows the kinetics of Pseudomonas aeruginosa deactivation with PAA, with and without UVC.

FIG. 19 shows the kinetics of Salmonella enterica deactivation with PAA, with and without UVC.

FIG. 20 shows the kinetics of Candida albicans deactivation with PAA, with and without UVC.

### PREFERRED EMBODIMENTS OF THE INVENTION

An environment to be decontaminated may be a room, for example, a hospital room or ward, a clinic, a hospital operating theatre, ambulance or patient transport, a workspace, a domestic room, a shipping container, an aircraft interior, warehouse, plant or animal production facility or other enclosed or semi-enclosed space. Exposed surfaces may be exemplified by surfaces of walls or partitions defining the environment or space, or work surfaces, machinery surfaces, air conditioning ducts, or other surfaces which are interior or can be enclosed or partly enclosed, at least temporarily, for the present purpose. Exposed surfaces also include the contents of the room, such as beds, bedding and pillows, chairs, tables, televisions and controllers and so on.

By decontaminating an environment it is meant that the air in the volume, along with any organisms suspended in the air, are subject to decontamination.

In the instant application, it is preferred that the environment or space to be disinfected is closed. By this it is meant that the space is to some extent sealed to minimise the amount of air replaced over time, but it is also important that the space is cleared of humans and other species that need to be protected during the decontamination cycle.

An environmental disinfection system according to many embodiments of the invention uses a combination of peracetic acid vapour and UV-C light to decontaminate a room. Both peracetic acid and UV-C are individually known as decontamination agents, however, the present applicants have found that surprisingly environments subjected to the simultaneous application of both peroxide vapour and UV-C light exhibit a synergistic reduction in the amount of bacteria present. It has also been found that when the peracetic acid and UV-C emanate from a single locus in a closed space, the synergistic effect is such that despite the differing modes of the UV-C and peracetic acid, a fairly uniform level of synergistic effect is observed within the entire closed space.

In many embodiments, a decontamination method comprises contacting a surface to be disinfected with a simultaneous combination of peracetic acid vapour and UV-C light.

In certain embodiments, a device of the present invention dispenses the peracetic acid vapour and UV-C light from a single combination device, with the peracetic acid and UV-C emanating from a single locus. Of course it should be appreciated that embodiments of the invention are not so limited; rather in many embodiments the peracetic acid and UV-C can issue from differing loci, or even from multiple loci spaced around the environment.

The peracetic acid vapour can be present in any amount, for example from 0.15 to 1.5 ppm. The overuse of peracetic acid can lead to the formation of a film of condensate on the surface which is unpleasant to the touch and generally regarded as undesirable.

Similarly, the UV-C can be used at any power level, although excess exposure to UV-C can lead to the damage of surfaces, particularly if they are organic based (cloth or wood) or plastic. A suitable UV-C power level can be from 0.5 to 20 mJ/cm², e.g. 1 to 15 mJ/cm², e.g. 5 to 10 mJ/cm².

It has been observed that even when very low amounts of peracetic acid and low UV-C intensities (e.g. 0.15 ppm PAA and 5.0 mJ/cm² UVC) are used, a synergistic effect is still observed. The combination of PAA vapour level and UVC intensities can give a synergetic effect for different bio-burden slightly in different ranges. The synergistic interaction as proposed has been observed over a wide range of peracetic acid concentrations and UV intensities.

One preferred device of the present invention is configured to dispense both UV-C and peracetic acid from a single locus. This is a standalone apparatus capable of disinfecting a typical sized hospital or clinic room. The typical target room can be about 9 m² (3 m×3 m) in floor area and with a standard height of about 2.6 to 3 m.

In order to carry out the decontamination, the room may be closed in the usual way that a domestic, clinic or hospital room would be closed off, namely by closing windows, doors and external vents and turning off any air-conditioning. However, the room does not need to be hermetically sealed. A small amount of airflow was permissible, provided that the airflow does not result in more than one air change per hour.

The peracetic acid vapour generator according to certain embodiments may be typically positioned on one wall, at an elevated position (e.g. at a height of between 1 to 1.5 m above the floor), such that the peracetic acid can be directed outwardly along the room, towards the opposite wall without obstruction. The UV-C source may also directed across the room and downwards such that the human contact surfaces in the room are exposed to it. Alternative positions and arrangements are also envisaged, for example, multiple devices could also be used in larger spaces on different walls or positioned on the ceiling separately or in clusters (2 or more, e.g. 2 to 5) to achieve an appropriate distribution of peracetic acid vapour and UV-C.

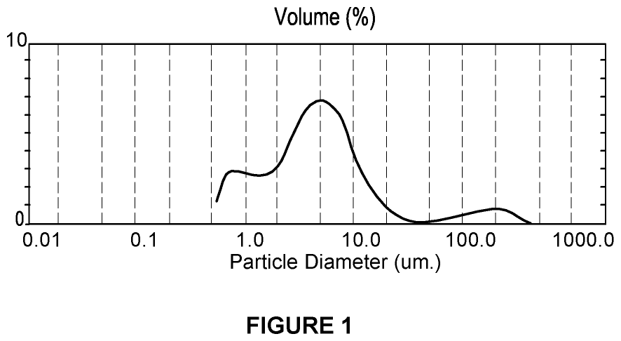

In one embodiment, an ultrasonic nebulizer is used to sonicate a peracetic acid solution contained within the device. The typical droplet size distribution for ultrasonic nebulizer depends on the vibrating frequency of the ceramic disc. Ultrasonic nebulizers incorporate a piezoelectric crystal vibrating at high frequencies (for example 1-3 MHz) in order to produce aerosol. The ultrasonic nebulizer can operate at any suitable frequency. A commercially available 2.4 MHz transducer, for example, can be used. In certain embodiments, once the mist is generated, it is transferred to the outside of the device by way of a fan operating at sufficient speed to propel the droplets to the outside of the device. The size distribution of the mist produced by a 2.4 MHz ultrasonic nebulizer is shown in FIG. 1. A logarithmic scale is used to accommodate the wide range of droplet sizes.

In the illustrated example, the droplet size was as follows:
Most common droplet size=4.47 μm
Volume of droplets 1 μm or less=14%
Volume of droplets 1 μm to 4 μm=40%
Volume of droplets 4 μm or less=54%
Maximum droplet size detected=350 μm

Theoretically the average droplet diameter (d) is dependent on the forcing frequency (f) and the fluid properties surface tension (ρ) and density (a) (both temperature dependent) and is governed by the following equation.

$$d \approx \text{.34} \sqrt[3]{\frac{8 πσ}{ρ {f}^{2}}}$$

The vertical scale represents the % of the total droplet volume occurring a particular diameter. As the volume is proportional to the cube of the diameter, it takes 1 million droplets at 2 μm to equal one droplet at 200 μm. This means that although the % volume around 200 μm looks significant it represents a very small % of the total number of droplets produced.

Small variations in transducer frequency and transducer strength had no material effect upon the operation for the device.

The inventors found that some degradation of peracetic acid occurs when an ultrasonic nebulizer is used. Without wishing to be bound by theory, it is believed that this is because the ultrasonic energy causes the peracetic acid solution (from which the mist is generated) to rise in temperature.

Accordingly, in many embodiments, a pneumatic nebulizer or a mesh nebulizer may be implemented. The usage of these nebulizers may not result in the same level of degradation of peracetic acid.

In many embodiments, a pneumatic nebulizer (also known as a jet nebulizer) is used to generate the peracetic acid mist from a peracetic acid solution.

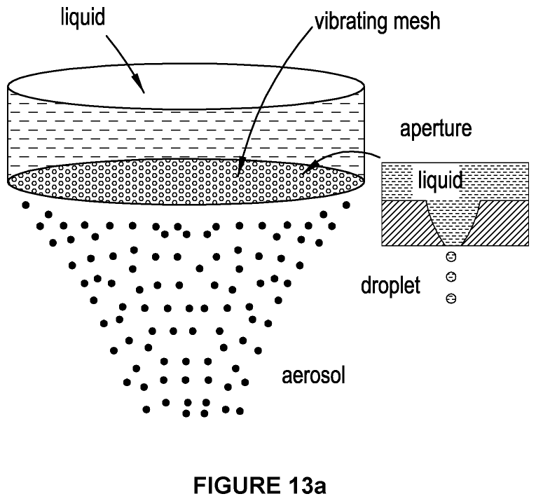

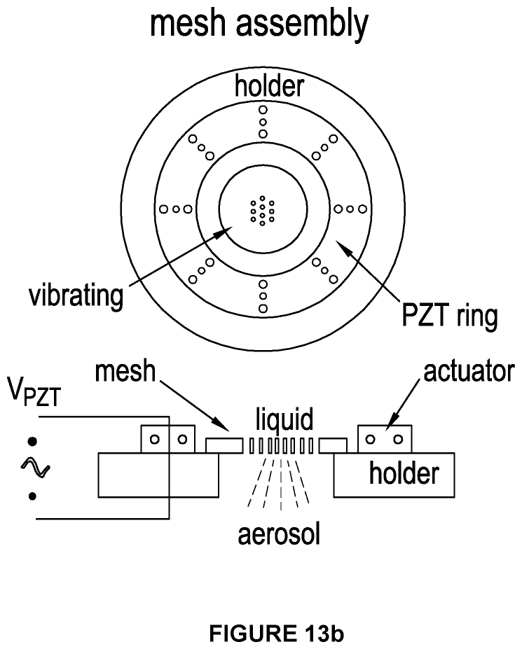

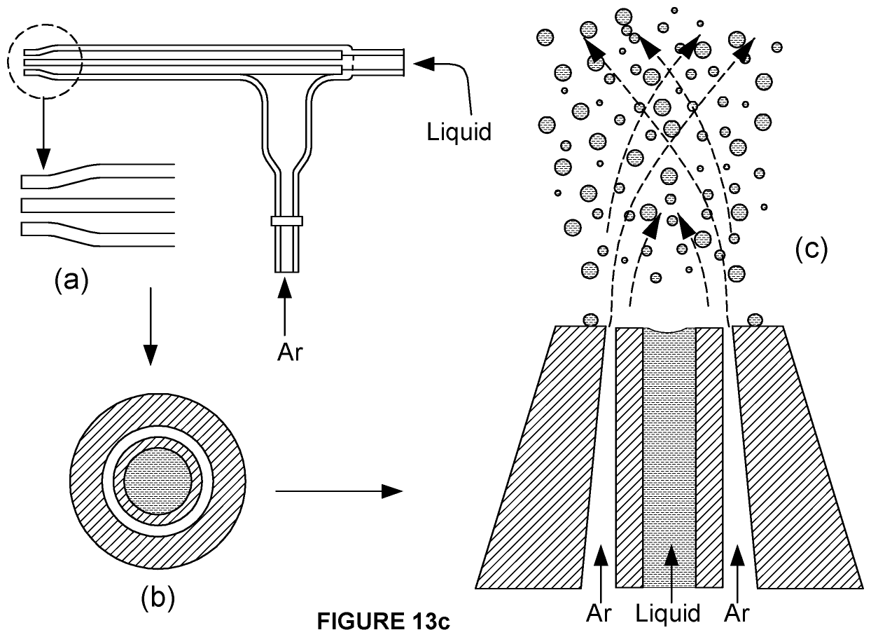

An example of a pneumatic nebulizer assembly is shown in FIG. 13c. FIG. 13c (a) shows the nebulizer including liquid inlet and the inlet for compressed gas (such as Argon (Ar) or compressed air) and a close-up of the nozzle in side view. FIG. 13c (b) shows the front view of the nozzle shape and the relative positioning of the outlet for the liquid (the inner circle) and the outlet for the compressed gas (a ring shape). FIG. 13c (c) shows generation of a mist from liquid at the nozzle during the nebulization process. Alternatives to Ar can be used, e.g. compressed air.

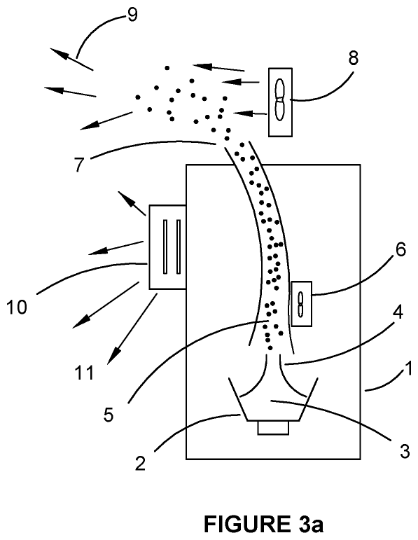

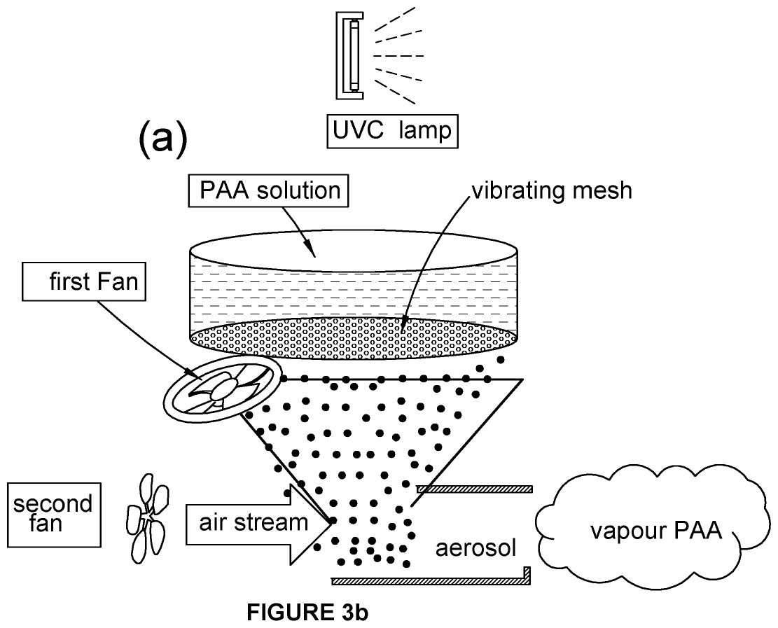

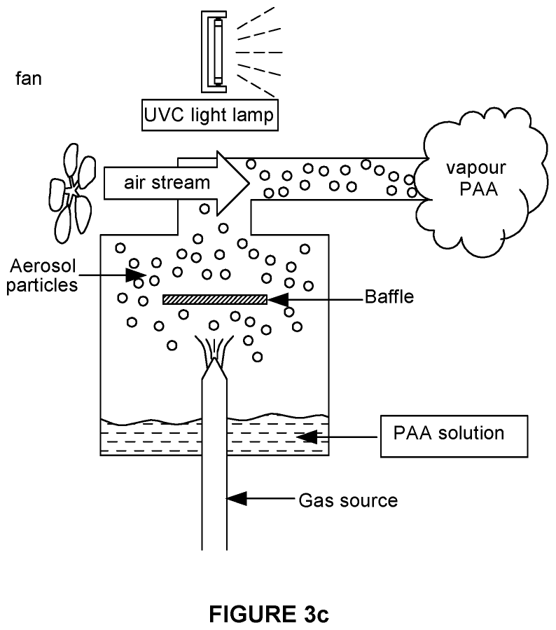

An example of a system implementing a pneumatic nebulizer is shown in FIG. 3c. The gas source in FIG. 3c can be a source of compressed air. The baffle is used to form mist as the gas and liquid passes through it.

The pneumatic nebulizer does not require a ‘first fan’—it can utilize the compressed air to direct the flow of the mist. A fan can be used to generate the vapour from the mist and propel the vapour across the room.

A pneumatic nebulizer uses a pressurised gas (preferably air) to generate the mist. However, the inventors have found that the ratio of components (i.e. hydrogen peroxide, acetic acid and peracetic acid) in the mist may not be the same as the ratio of components in the peracetic acid solution. Without wishing to be bound by theory, it is believed that this is due to the differences in boiling points.

In typical nebulizer assemblies any oversized droplets (e.g. 50 micrometers or more) are filtered out mechanically using baffles and/or tortuous pathways. However, in certain embodiments of the present invention, the removal of oversized droplets is achieved in the relatively large space above the nebulizer where the air carrying the mist slows down, thereby allowing the large droplets to fall out (e.g. droplets of size 50 micrometers or more). Thus, the device can produce a mist immediately adjacent the device which is relatively free from large droplets (e.g. droplets of size 50 micrometers or more).

In a further embodiment, a mesh nebulizer is implemented.

In a number of embodiments, a mesh nebulizer is used to generate the peracetic acid mist from a peracetic acid solution. A mesh nebulizer involves generating a mist from a liquid using a vibrating mesh by pushing a small volume of liquid through the mesh.

An example of a mesh nebulizer assembly is shown in FIG. 13a and FIG. 13b. The nebulizer in one embodiment is a PZT nebulizer that has high resonant frequencies of 102.9 kHz and 103.8 kHz.

An example of a system implementing a mesh nebulizer is shown in FIG. 3b. In FIG. 3b, an aerosol is generated from a peracetic acid solution using the mesh nebulizer. A first fan propels the droplets to a location adjacent the device and a second fan evaporates the peracetic acid droplets to peracetic acid vapour and propels the vapour away from the device.

An advantage of using a mesh nebulizer is that the resultant mist has a uniform droplet size. The droplet size is dictated by the size of the holes in the mesh. For example, holes in the mesh (and hence the droplet size) may be 10 micrometres or less (e.g. 8 micrometres, e.g. 5 micrometres). A vibrating mesh nebulizer has a narrow, very small, particle size distribution, which helps to speed up evaporation process and avoid wetting disinfecting surfaces by large droplets. A further advantage of using a mesh nebulizer is that it avoids uncertainty regarding the peracetic acid concentration in the droplets of the mist. In other words, the ratio between the components in the vapour will be the same as in the solution.

While certain pneumatic nebulizers and mesh nebulizers have been mentioned, it should be clear that any suitable pneumatic or mesh nebulizers may be implemented in accordance with embodiments of the invention.

In a number of embodiments, when the mist of peracetic acid droplets exits the device, the droplets are directed into the path of fan (e.g. a second fan). The droplets are immediately contacted by the airflow of the second fan which serves the dual purposes of i) vapourizing the droplets entirely and ii) setting up a flow sufficient to propel the vapour to the opposite side of the room. The second fan can be considerably more powerful than the first, and the power chosen so that sufficient propulsion of the vapour across the room is achieved. In one embodiment, the difference between air flow rate from the nebulizer and “spreading” fan was more than 100 times.

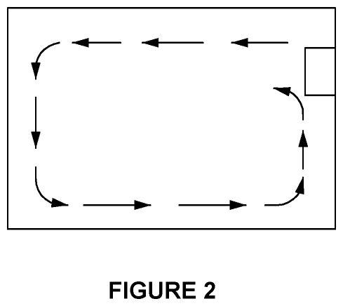

The flow of peroxide vapour under these conditions may occur as illustrated in FIG. 2, namely, in a cyclonic flow, from the device across the top half of the room to the opposite wall, dropping downwardly, and then returning along the lower half of the room towards the device.

As alluded to previously, any suitable nebulizer may be implemented in accordance with embodiments of the invention. The mist need not be produced by an ultrasonic nebulizer but could be produced by any suitable nebulizer, for example, a pneumatic nebulizer or a mesh nebulizer. Likewise, any type of fan could be used, for example, the fan could be a conventional fan or a bladeless fan or the fan could even be replaced by compressed air.

The nebulizer and first and second fans may act in combination to produce a peroxide mist which is vapourised immediately upon exiting the device. If the mist particles are nebulized to be too large, or the second fan does not provide adequate air flow, the large droplets may be propelled away from the device by the second fan, thereby impeding the evaporation of water and formation of vapour. Mist particles can potentially settle on surfaces and produce an undesirable wet feel. Particles may also be less effective than vapour in contacting the undersurfaces of horizontal surfaces or in reaching less accessible areas by tortuous paths. As mentioned above, many embodiments of the present invention can provide a space adjacent the device where large droplets in the mist can drop out of the mist prior to the mist being vapourised.

Likewise, if the nebulizer produces particles that are too small, evaporation may occur prior to the mist particles exiting the device, thereby resulting in potentially inconsistent results.

FIG. 3a shows an embodiment of a device 1 for the preparation and dispensation of peracetic acid vapour. The peracetic acid generator 1 employs an ultrasonic nebulizer 2 to sonicate a peracetic acid solution 3. This sonication produces an ultrasonic fountain 4 which in turn generates a finely dispersed mist 5 of peracetic acid droplets. The finely dispersed mist 5 of peracetic acid droplets is propelled from the nebulizer 2 and passes through outlet 7 by way of a fan 6. The mist exits the device into the airstream of a second fan 8, which is a part of the device. The second fan 8 serves to evaporate aerosol droplets to produce vapour 9. The second fan also serves to create a flow current to propel the vapour 9 across the room.

The peracetic acid vapour was generated from a commercially available 5% w/w peracetic acid solution. Peracetic acid is sold as an equilibrium mixture of peracetic acid, hydrogen peroxide, acetic acid and water. The 5% w/w peracetic acid solution comprised typically 20-30% w/w hydrogen peroxide, 6-10% w/w acetic acid 4.5-5.4% w/w peracetic acid and the balance being water. Higher concentrations of peracetic acid solution, for example, up to 10% w/w or up to 15% w/w can be used. Indeed, any suitable concentration may be used in accordance with embodiments of the invention.

The peracetic acid vapour generator in FIG. 3a, 3b or 3c may be operated in combination with a UV-C light 10 located in the same housing. The UV-C lamp emits UV light 11 at a wavelength of 100-280 nm (e.g. preferably 200-280 nm, 220 nm to 270 nm, 240 nm to 260 nm, about 250 nm), typically about 254 nm. The light is chosen to provide a sufficient intensity (from 5 to 25 mJ/cm², e.g. from 5 to 15 mJ/cm² or from 15 to 25 mJ/cm²) on the remote areas of the room. The UV light may be shielded (e.g. using a quartz shield such as synthetic quartz) to prevent the formation of ozone.

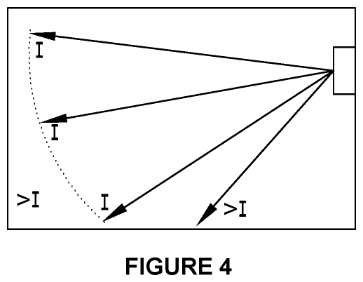

The distribution of UV from a point source can result in light of differing intensities hitting different surfaces within the closed environment. The light intensity decreases in accordance with the inverse square law, so surfaces further from the light may receive lower intensity. However, the angle of incidence of the UV-C light upon the surface can also influence the intensity of the light. FIG. 4 shows the UV light irradiating a room.

A low pressure mercury-vapour lamp can be suitable to generate the desired light of the desired wavelength, although any light source generating light of a suitable wavelength or intensity can be used, for example, UV-C LEDs. The use of UV-C LEDs allows for fine control of the delivered irradiance/UV component of the disinfection algorithm due to the control of the light output from LEDs.

The combination of UV-C and peracetic acid surprisingly resulted in synergy against a number of bacteria, including Clostridium difficile. The kill against those organisms was demonstrably much higher than for the individual elements of UV-C or peracetic acid, and even significantly higher than would be expected for the two individual elements, thereby demonstrating significant synergism between the UV-C and the peracetic acid.

Because of the nature of the two disinfectants, i.e. chemical (peracetic acid) and energetic (UV-C), the distribution of each source cannot be such that the concentration and intensity are experienced evenly at every point in the room. For instance, some areas experience more peracetic acid vapour and less UV-C, in other areas, the reverse will be true. Some regions will receive less of both and there will also be expected to be one or more “sweet spots” where both the peracetic acid and UV-C are high.

However, it was surprisingly found when mapping the UV intensity and peracetic acid flows that the amounts of each delivered to most points in the room were, largely complementary. That is, while there were some regions receiving larger and smaller doses of UV-C and peracetic acid, overall, the effect largely covered the entirety of the room in a very satisfactory manner.

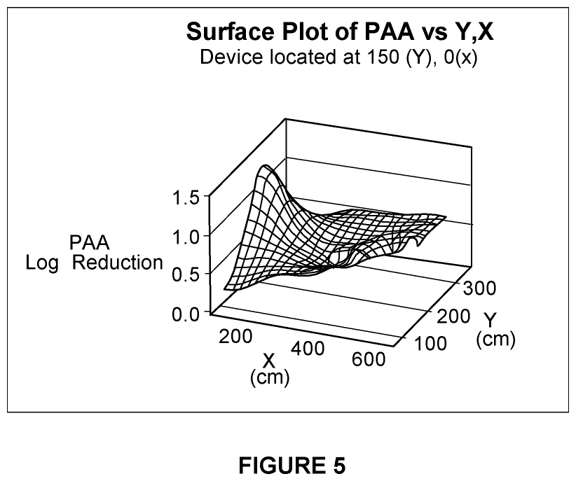

FIG. 5 shows a surface plot of the disinfective ability of peracetic acid throughout the room, as an xy plot dependent upon the floor location, based upon both UV-C and peracetic acid issuing from a single locus. There was a high level of disinfection below the peracetic acid generator, and an increased level along the wall opposite the peracetic acid generator, with a slight trough near the middle of the room.

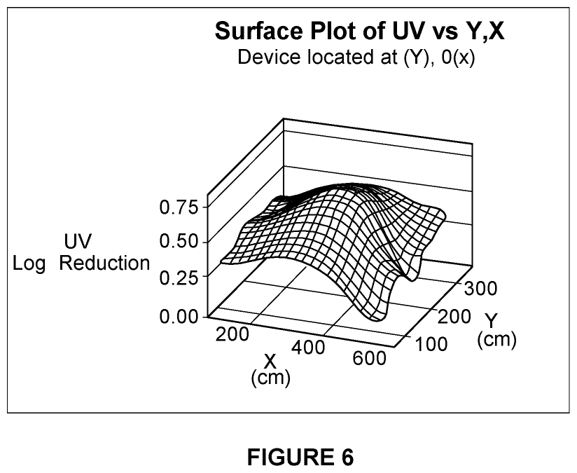

FIG. 6 showed a surface plot of the disinfective ability of UV-C throughout the room, as an xy plot dependent upon the floor location. There was a significant drop off in disinfective ability at the far wall, as expected and a high level in the middle of the room. Some shading by the device itself was responsible for a decrease immediately below the sterilizer.

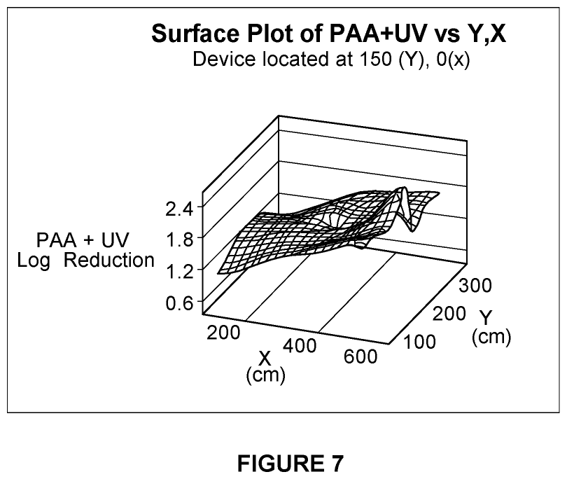

FIG. 7 shows a surface plot of the disinfective ability of UV-C and peracetic acid in combination throughout the room, as an xy plot dependent upon the floor location. Even though the combination of UV light and peroxide vapour concentration are not experienced evenly over the room, it can be seen that the level of disinfection provided was significant across the entire room, and surprisingly uniform, with no areas having an unacceptably low level of disinfection.

FIG. 7 shows a region towards the centre of the room where the demonstrated synergy provides the maximum benefit, which could be termed a “hot-zone” within the decontamination area. It is believed that this is due to optimal and unshadowed UV-C exposure in combination with contact with a relatively undepleted peracetic acid vapour stream.

In a number of embodiments, the method of decontaminating an environment or surface comprising the step of simultaneously subjecting said environment or surface to a combination of peracetic acid vapour and UV-C light further comprises a humidification step. The humidification step may be carried out before the decontamination step and/or simultaneously with the decontamination step.

In accordance with certain embodiments of the invention, the humidity of the environment or room may be adjusted to 55% or higher (e.g. 60% or higher, e.g. 65% or higher, e.g. 70% or higher). The humidity may be 95% or less, e.g. 90% or less, e.g. 85% or less. In one embodiment, the humidity of the environment or room is preferably adjusted to a level in the range of from 60 to 85%.

The biocidal efficiency of PAA vapour can increase with increasing humidity level. This PAA vapour behaviour is exactly opposite from the behaviour of hydrogen peroxide vapour, which is a more effective disinfectant at low humidity level.

The inventors have found that decontamination at a higher humidity proceeds more efficiently. Therefore, a higher humidity level means that the decontamination can be carried out in a shorter time.

The decontamination process could be run for any desired time length (e.g. from about 5 minutes to about 24 hours, from about 30 minutes to about 4 hours, from about 30 minutes to about 3 hours). It was found to be advantageous to run the process for a period of about two hours. That produced a level of decontamination that would have a very significant clinical impact in terms of reduced levels of acquired infection. Two hours was also considered a relatively acceptable length of time to shut off a room in a hospital setting.

The combinatorial approach of the present invention can be very flexible to bio-decontamination requirements and at the same time safe for construction materials. It can be done by variation of PAA vapour level, applicable UVC dosage and disinfection time.

In some embodiments, disinfection is followed by remediation. Remediation refers to reducing the level of toxic components of the biocide.

In one embodiment, remediation comprises using the natural degradation of PAA that occurs over time.

In another embodiment, remediation comprises using existing air conditioning to remove and replenish the air inside the disinfected space.

In some embodiments, following disinfection, decontaminating devices can perform remediation of the disinfected space.

In a further embodiment, remediation involves use of a filter that can remove acidic vapour from the treated area, such as an activated carbon filter. Remediation may involve use of a catalytic cartridge comprising activated carbon.

In one embodiment, the activated carbon is optionally impregnated with an alkaline material (e.g. potassium hydroxide, e.g. Honeycomb Activated Carbon impregnated with KOH), which can absorb residual acidic vapour. This can destroy residual acidic vapour. Consequently, the associated smell of the acidic vapour is removed from the air in the room.

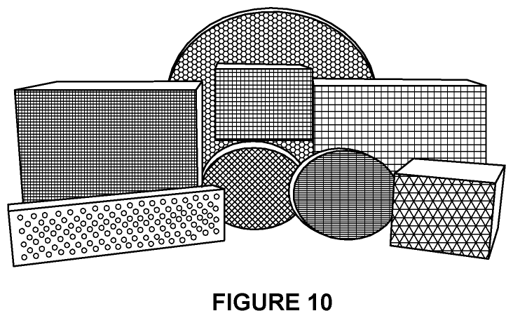

In one embodiment, remediation comprises catalytic destruction of toxic components of the biocide (PAA and H₂O₂) at room temperature. In this case, using specific Activated Carbon (AC) as a catalyst, H₂O₂ is converted to water and oxygen and PAA to acetic acid and oxygen. All these products are not toxic and are environmental friendly. AC impregnated with KOH can be very useful for removing acidic components of disinfectant due to quick irreversible chemical reaction between KOH and Acetic and Peracetic acids. Some shapes of ACH available now can be seen from FIG. 10.

The honeycomb structure of AC can avoid high back-pressure and can help to reduce remediation time.

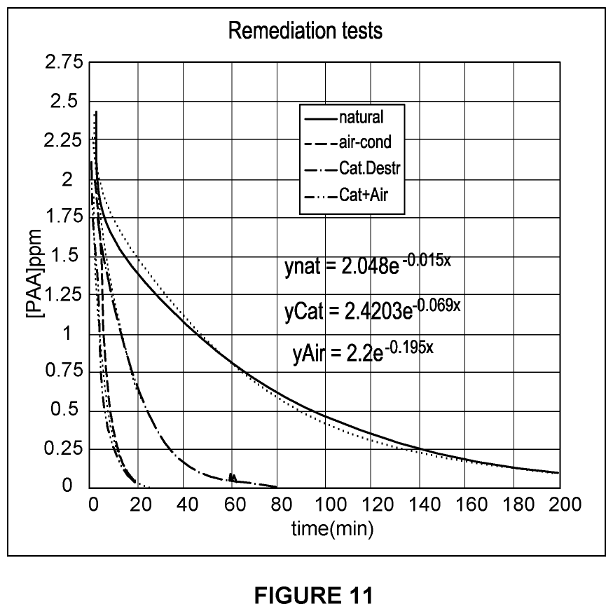

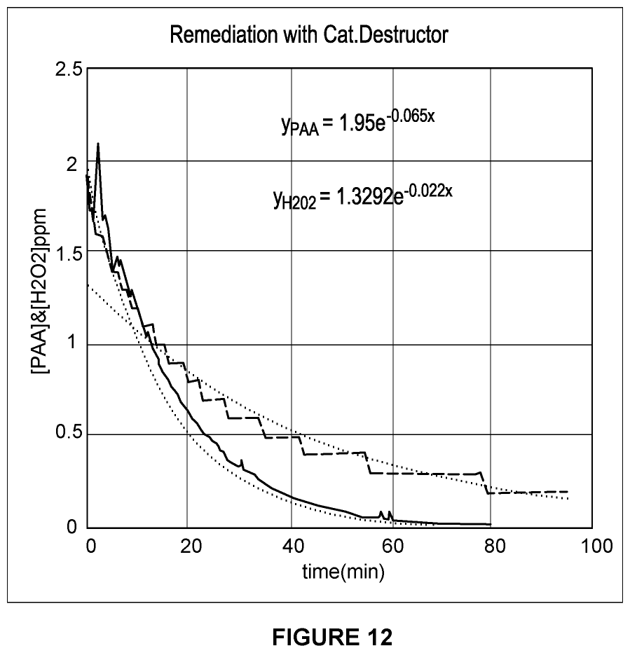

Results of tests comparing remediation using natural degradation of PAA, catalytic destruction with Honeycomb AC and using existing air-conditioning are presented in FIG. 11. As can be seen from FIG. 11 the most effective option for PAA removal is using air-conditioning. However, this option cannot be used in all cases due to distribution of toxic PAA vapour around the building through the air-conditioning system. The use of catalytic destruction and adsorption is more universal and in certain circumstances can be rather effective. Another aspect of the toxic chemical removal process using a catalytic cartridge relates to the difference in rate of H₂O₂ and PAA destruction (see FIG. 12). PAA can be removed more rapidly than H₂O₂. This is believed to be due to the significant difference in boiling temperature of these components (˜105° C. for PAA and 150° C. for H₂O₂).

At the end of the decontamination process, the device was switched off. The room was entered after a few minutes. There was no adverse smell and no residual residue could be observed by sight or touch on any surface, including organic and porous surfaces such as paper and cloth.

One “unpleasant” side of using PAA as biocide is the post-disinfection odour of acetic acid (vinegar). Acetic acid is a natural component of PAA solution and can also result from catalytic destruction of PAA. This odour problem arises due to the large gap between acetic acid vapour safety level, which is 10 ppm (TWA) and the threshold of odour detection, which is only 0.48-1.0 ppm. PAA which has the same odour as acetic acid will be removed to well below safety level (0.17 ppm) by a catalytic cartridge but unfortunately, the catalytic destruction involves converting PAA into acetic acid. The same cartridge is used for removing both PAA and acetic acid by adsorption but in either case a vinegar odour unfortunately still can be sensed at the end of disinfecting cycle due to the very low threshold scent for acetic acid. One solution to overcome this problem is adsorption of acetic acid by suitable adsorption cartridge and using masking agents after.

Optionally a masking agent may be used following disinfection. Many commercial odour control products are available on the market. Usually they are mixtures of essential oils with addition of surfactants to disperse the essential oil components in water solution.

Neutralising odours with essential oils is a physical-chemical reaction, which relies upon the ‘Van Der Waals’ principle. The odour molecules of the active substance in the vapour phase react with the scented molecules. In that way, most of the scented molecules are directly converted into unscented molecules. Thereby 70-90% of the smell is aborted. Any remaining odour is neutralized by the Zwaardemaker-principle, so that the scented molecules are no longer perceived by the nose.

Devices in accordance with many embodiments of the present invention can be fitted with a timer to ensure a predetermined time of operation.

Alternatively, the devices can be equipped with a motion and or thermal detector such that they are capable of autonomously disinfecting a room for periods when it is empty.

Disclosed devices may also be integrated with systems such as building climate and security systems and patient and staff management systems to automate cleaning during downtimes.

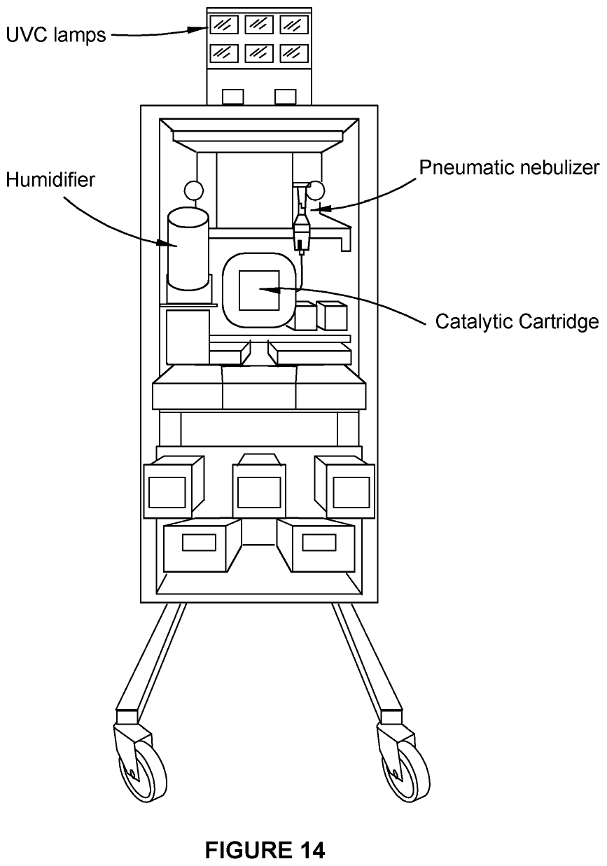

FIG. 14 shows a prototype of a device comprising a nebulizer, UVC lamps, a humidifier and a catalytic cartridge. The prototype includes the following sub-systems:
1. Humidifier with automatic control Humidity level:
2. PAA vapour supply sub-system using a pneumatic nebulizer. Construction of pneumatic nebulizer was designed such a way to produce “dry mist”, which immediately converts into PAA vapour at room temperature.
3. Catalytic and Adsorption converter to speed up PAA and Hydrogen peroxide destruction at the end of the disinfection cycle as well as removal of VOC if needed. For effective remediation, Honeycomb Activated Carbon impregnated with KOH may be used.
4. Sub-system for supplying vapour of masking agent based on the same principles as PAA supply sub-system using a pneumatic nebulizer.
5. UVC lamp protected with UVC-transparent quartz cover.

### Experimental

A room of volume 66 m³ (6 m×4.2 m (floor area)×2.6 m (height)) was used as a test room. The room was closed in the usual way, that a domestic, clinic or hospital room would be closed off by closing windows, doors and external vents and turning off any air-conditioning. However, the room was not hermetically sealed and a small amount of airflow was permissible. The temperature of the test room was ambient room temperature (between 20° C. and 25° C.).

The test room was somewhat larger than that which would normally be targeted, which is a room of 3×3 m floor area by 2.6 to 3 m high.

A peracetic acid vapour/mist generator (nebulizer) was positioned along the smaller wall, at a height of about 1.21 m.

The peracetic acid vapour/mist generator utilised a commercially available 5% w/w peracetic acid solution. Peracetic acid is sold as an equilibrium mixture of hydrogen peroxide, acetic acid and water. 5% w/w Peracetic acid solution typically comprises 20-30% w/w hydrogen peroxide, 6-10% w/w acetic acid 4.5-5.4% w/w peracetic acid and the balance being water. In many embodiments of the present invention, the 5% w/w peracetic acid solution used comprised 24% w/w hydrogen peroxide, 7.5% w/w acetic acid 5% w/w peracetic acid and the balance being water.

The peracetic acid vapour/mist generator was also accompanied by a UV light that was located in the same housing. The UV lamp was a Philips TUV PL-L 18 W/4P 1CT/25 which operated at 60V/0.37 A/18 W and had a UV-C power output of 5.5 W. The lamp is a low pressure mercury-vapour lamp emitting a peak about 254 nm (253.7 nm), a known germicidal wavelength. That is a known germicidal wavelength. The UV light shone upon all the exposed surfaces in the room. The UV lamp was 18 watts. The bulb used synthetic quartz to prevent escape of 185 nm ozone-forming radiation.

UV-C light intensity falls off in accordance with the inverse square law, so that areas located further from the light are subjected to significantly less intensity. The high location and position of the sterilizer means that the room boundaries most closely approximate the same distance from the UV lamp.

The UV-C light and peracetic acid vapour/mist generator could be operated independently or simultaneously, allowing for a direct comparison of the individual decontaminating agents with each other and with the combination.

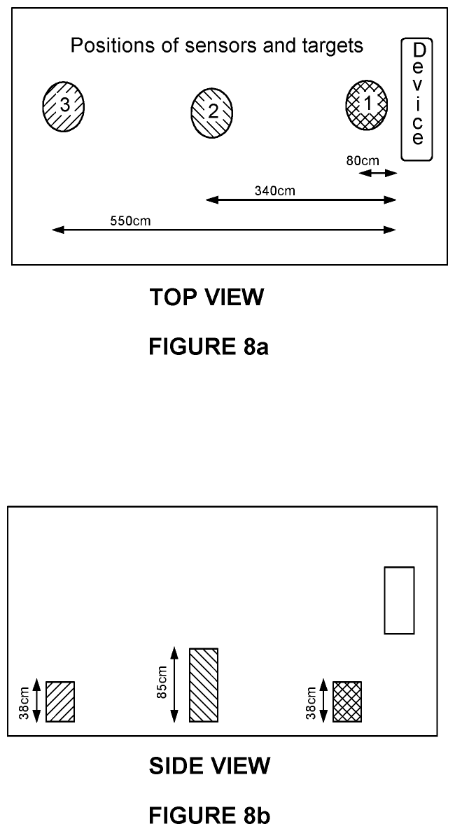

Immediately before commencing sterilization, petri dishes containing inoculum were placed at three discrete locations within the room—one close to the UV/peracetic acid device (e.g. 80 cm from the device), one in the middle of the room and the other at the opposite end of the room. The dishes were also placed at differing heights. The positions of the dishes are shown in FIGS. 8a and 8b. The petridishes were “dirty” and contained 5% horse serum. Each dish had an inoculated area of 50 mm of Clostridium difficile ATCC43539. This was prepared by spreading 100 μl of microorganism over 50 mm surface area on the treated petri dish and allowing them to dry. The number of microorganisms was counted. Standard diluents (synthetic broth) were used when preparing cultures. Plates were incubated as required.

A control plate prepared in an identical manner and having the same level of Clostridium difficile ATCC43539 was placed outside the room, but in otherwise similar conditions.

The control plate outside the disinfection room behaved as expected. The initial number of Clostridium difficile was 2.45E+03 per 50 mm which increased slightly over the course of two hours to 2.90E+03. That is, after 2 hours without any disinfection, Clostridium difficile showed an increase of 4.5E+02.

The UV light and peracetic acid vapour/mist generator was operated to generate UV light only, with no peracetic acid mist, for a period of two hours. The Clostridium difficile plates located in the test room showed either a modest reduction (a 0.11 log reduction) or a zero change in Clostridium difficile numbers (i.e. a biostatic treatment). The biostatic result was observed on the plate furthest from the UV source, which was also subjected to some shadowing.

The extent of UV sterilization showed that the amount of bactericidal effect achievable was minimal at positions 1 and 2, closest to the UV light, with no effective sterilization (but a bateriostatic effect) at position 3. Position 1 was shadowed.

In a repeat experiment, the UV light and peracetic acid vapour/mist generator was operated to generate peracetic acid vapour only, with no UV light emanating, for a period of two hours. The Clostridium difficile plates located in the test room showed a reduction in microorganisms ranging from a 0.71 log reduction to a 1.19 log reduction. The efficacy of the peracetic acid surprisingly was highest on the plate located furthest from the peracetic acid source. Without wishing to be bound by theory, it is believed that this may be due to current effect, in which the vapour is propelled along the top of the room before moving downwards on to the wall and travelling back along the floor. vapour concentration would be expected to be higher at the base of the wall opposite the sterilizer.

The experiment was then repeated with both the UV light and peracetic vapour generator operating for a period of two hours. A very surprising result was obtained for the combined effect of both UV and peroxide. A marked synergism between UV and peracetic acid was observed. The Clostridium difficile plates located in the test room showed a reduction in microorganisms of between 2.11 log and 3.58 log, which was far in excess of any effect that could be predicted from the efficacies of the individual factors.

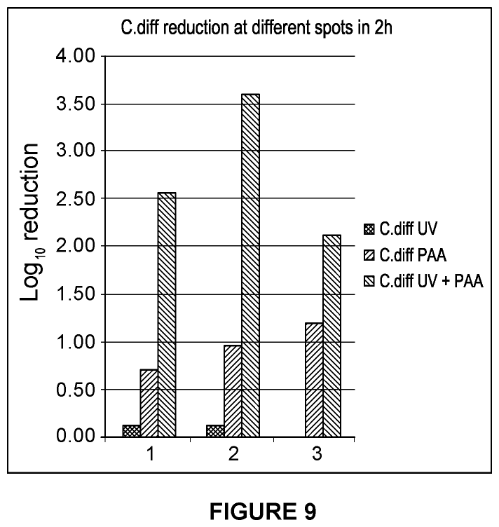

The results are shown in table 1 and presented graphically in FIG. 9.

TABLE 1
Control, UV-C, Peracetic acid and UV-C + Peracetic acid results

| | Control | Location 1 | Location 2 | Location 3 |
|---|---|---|---|---|
| Control | Log 2.6 | | | |
| | increase | | | |
| UV-C only | | Log 0.11 | Log 0.11 | No change |
| | | reduction | reduction | |
| Peracetic Acid | | Log 0.71 | log 0.97 | log 1.19 |
| only | | reduction | reduction | reduction |
| UV-C + Peracetic | | log 2.58 | log 3.58 | log 2.11 |
| acid | | reduction | reduction | reduction |

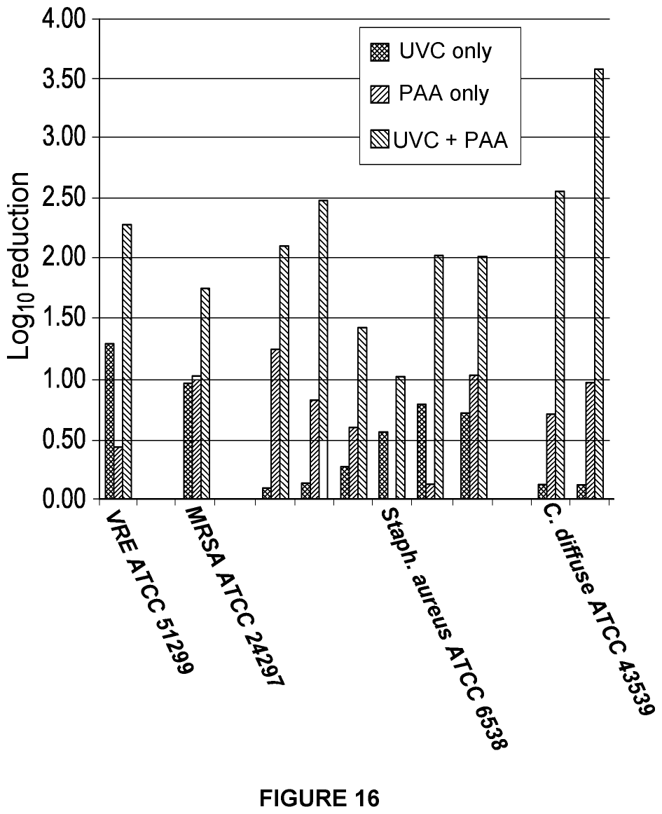

FIG. 16 shows the log 10 reduction with UVC only, with PAA only and with UVC in combination with PAA.

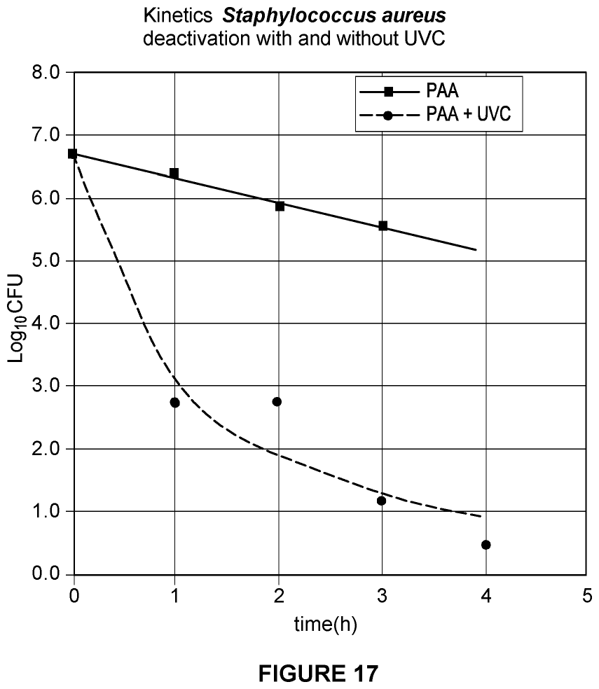

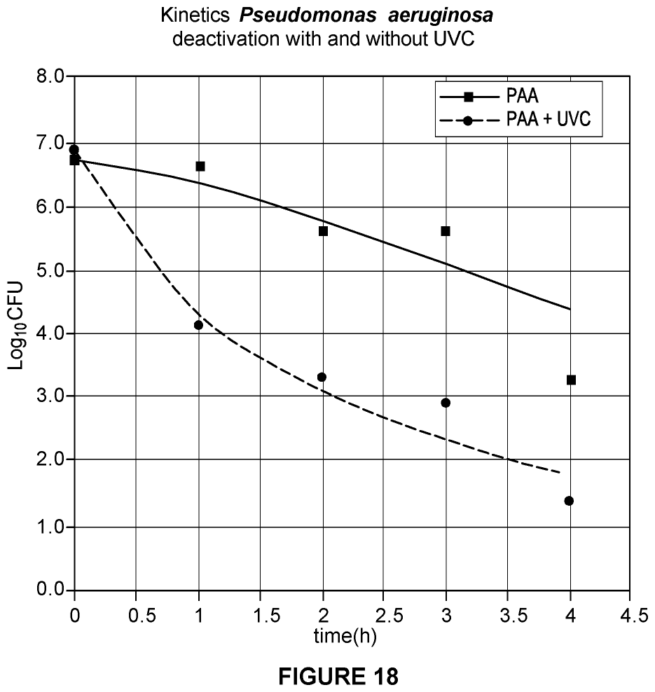

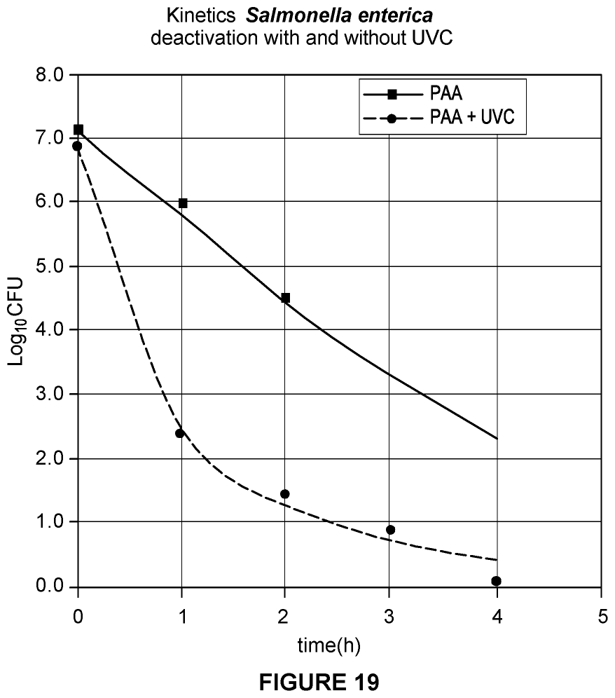

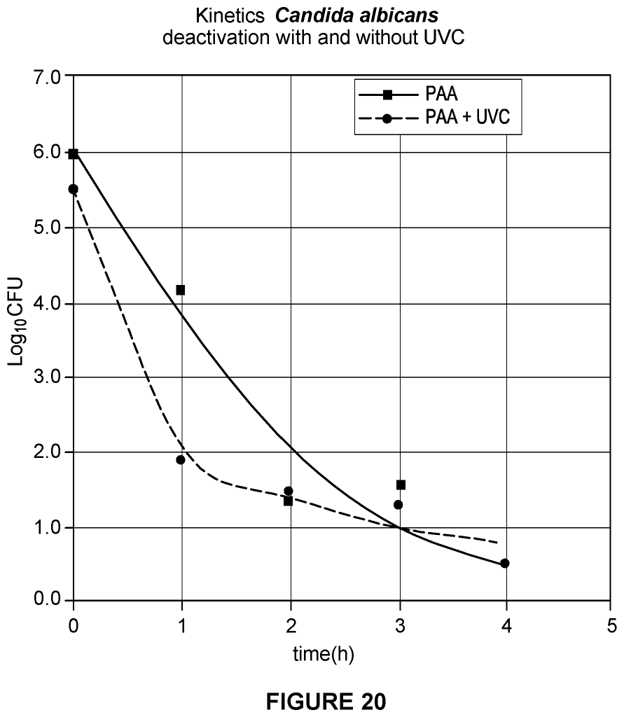

Kinetics deactivation for different bioburden are presented in FIGS. 17, 18, 19 and 20. FIG. 17 shows the kinetics of Staphylococcus aureus deactivation with and without UVC. FIG. 18 shows the kinetics of Pseudomonas aeruginosa deactivation with and without UVC. FIG. 19 shows the kinetics of Salmonella enterica deactivation with and without UVC. FIG. 20 shows the kinetics of Candida albicans deactivation with and without UVC.

In most cases, especially for the PAA+UVC combination, they are nonlinear in coordinates log₁₀[CFU]:-t. Formally it means that it was not a classical first-order reaction. The first-order model assumes that the cells and spores in a population have the same resistance and that the relationship between the decline in the number of survivors over the treatment time is linear. The model can be written as log₁₀(N/N₀)=−kt, where N₀ is the initial number of cells (CFU/ml), N is the number of survivors after an exposure time t (CFU/ml), k is the rate parameter, and t is the treatment time (min). The D value, a typical index of inactivation kinetics, is the decimal reduction time in minutes (time required to kill 90% of the organisms).

The Weibull model assumes that the survival curve is a cumulative distribution of lethal effects. The cumulative form of the Weibull distribution is given by log₁₀(N/N₀)=btⁿ, where b and n are the scale and shape parameters, respectively. When the shape parameter n is greater than or less than 1, the shape of the survival curve will present a shoulder or tailing, respectively. When n=1, the cumulative form of the Weibull distribution reduces to the first-order rate equation. For the Weibull model, the parameter t_{d}, which is analogous to the traditional D value, can be determined. t_{d} is the time required for d log reductions of the number of microorganisms and was calculated by the parameters b and n as shown in the equation

t_{d}=(In(0.9)/bx2.303)^{1/n}

where d is the desired log reduction, b=k and 2.303 is coefficient due to the shift from base e logarithms to base 10 ones.

All parameters of the Weibull Kinetic model for deactivation of all bioburden are summarised in Table 2:

TABLE 2
Parameters of Weibull kinetic model for deactivation of different bio-burden.

| | k_{PAA} | k_{PAA+UVC} | | | D_{PAA} | D_{PAA+UVC} |
|---|---|---|---|---|---|---|
| Bioburden | (h⁻¹) | (h⁻¹) | n | n | (min) | (min) |
| Staphylococcus aureus | 0.055 | 0.76 | 1.13 | 0.69 | 223 | 11 |
| Pseudomonas aeruginosa | 0.052 | 0.46 | 1.5 | 0.76 | 168 | 26 |
| Salmonella enterica | 0.2 | 1.02 | 1.24 | 0.72 | 70 | 8 |
| Candida albicans | 0.45 | 0.98 | 1.25 | 0.50 | 37 | 4 |

It is clear that the combinatorial approach dramatically improves bio-decontamination efficiency. For different bioburden, the difference in deactivation constants (k) can be more than 10 times. Differences in deactivation constant ratio for different bioburden are probably reflection of fluctuations in sensitivity of different bioburden to UVC and PAA exposure.

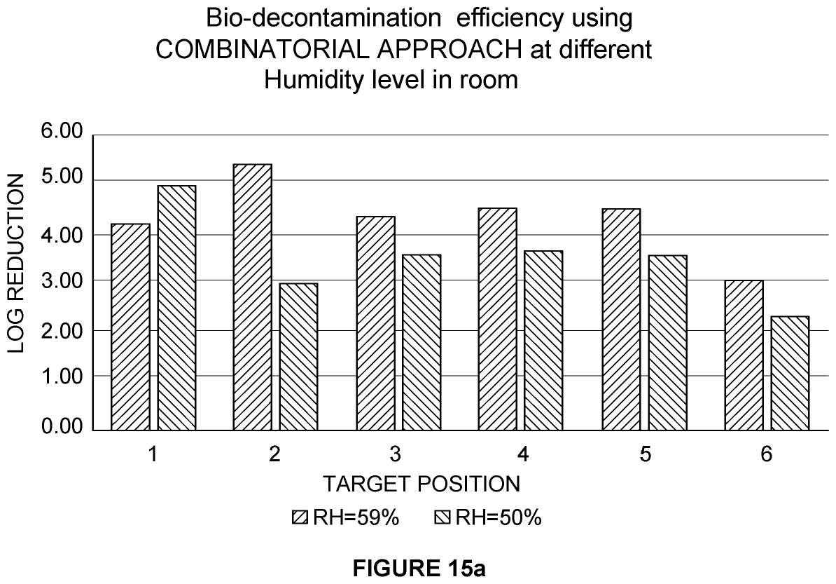

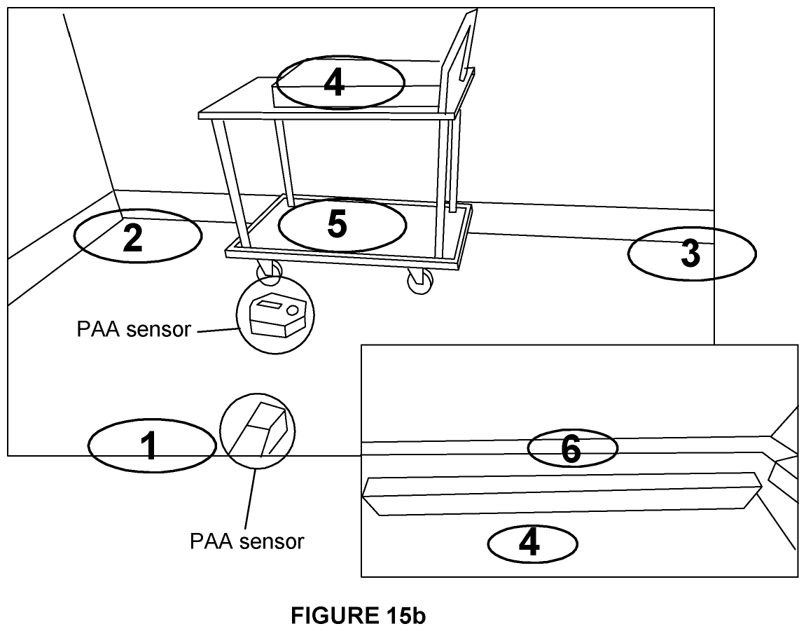

FIG. 15a shows bio-decontamination efficiency using a combinatorial approach of PAA and UVC at different humidity levels in the testing room. FIG. 15a shows the higher efficiency at 59% RH (relative humidity) compared with 50% RH. The positions of targets 1 to 6 in the testing room (˜25 m³) are shown in FIG. 15b.

In FIG. 15b, target 1 is positioned closest to the device. The device (not visible in 15b) is positioned on the opposite side of the room from target position 6. Target positions 5 and 6 are in shadow, i.e. an obstruction is present between the device and the target position.

The targets 1 to 6 with bioburden were glass slides inoculated with Staphylococcus aureus ATCC 6538. (the most resistant bio-burden from preliminary tests) with addition of Horse serum (dirty conditions).

FIG. 15a shows a greater log reduction at target positions 2 to 6 using the combinatorial approach of PAA and UVC at 59% relative humidity compared with 50% relative humidity. FIG. 15a shows a smaller log reduction at target position 1 using the combinatorial approach of PAA and UVC at 59% relative humidity compared with 50% relative humidity. In this regard, it is noted that the levels of PAA/UVC at target 1 were minimal due to its positioning.

1-34. (canceled)

35. A device, comprising:
a pneumatic nebulizer configured to produce a mist of peracetic acid droplets;
a mechanism to evaporate the peracetic acid droplets to peracetic acid vapour;
a mechanism to propel the peracetic acid vapour away from the device in a predetermined direction; and
at least one UV-C source configured to project UV-C radiation in the predetermined direction.

36. The device of claim 35, wherein the pneumatic nebulizer comprises:
a liquid inlet connected to a source of liquid peracetic acid,
wherein the liquid inlet is configured to receive, at a given time, only a discrete amount of the liquid peracetic acid for generation of the mist of peracetic acid droplets.

37. The device of claim 36, wherein the mechanism to propel the peracetic acid vapour away from the device comprises a gas inlet connected to a compressed gas source configured to receive a flow of compressed gas from the compressed gas source.

38. The device of claim 37, where the compressed gas is compressed air.

39. The device of claim 37, wherein the compressed gas is compressed Argon.

40. The device of claim 35, wherein the device is configured to produce a dry mist peracetic acid droplets that substantially immediately converts into the peracetic acid vapour at room temperature.

41. The device of claim 35, further comprising at least one of a fin, a blade, a louver, or a jet to provide directional flow of the peracetic acid vapour.

42. The device of claim 35, wherein the mechanism to propel the peracetic acid vapour away from the device comprises a fan.

43. The device of claim 35, wherein the pneumatic nebulizer includes a nozzle comprising at least one circumferential outlet for release of the mist of peracetic acid droplets.

44. The device of claim 43, where the at least one circumferential outlet comprises two or more circumferential outlets.

45. The device of claim 35, further comprising a humidifier.

46. The device of claim 35, wherein the at least one UV-C source comprises one or more UV-C lamps.

47. The device of claim 35, wherein the at least one UV-C source comprises one or more UV-C light emitting diodes (LEDs).

48. The device of claim 35, further comprising:
a catalytic cartridge.

49. The device of claim 35, wherein the mechanism to evaporate the peracetic acid droplets to peracetic acid vapour and the mechanism to propel the peracetic acid vapour away from the device in a predetermined direction are the same mechanism.

50. A method, comprising:
receiving, from a liquid peracetic acid source, an amount of peracetic acid solution via a liquid inlet of a pneumatic nebulizer;
at the pneumatic nebulizer, nebulizing the peracetic acid solution to an aerosol of peracetic acid droplets;
at the pneumatic nebulizer, evaporating the aerosol of peracetic acid droplets to peracetic acid vapour;
propelling the peracetic acid vapour away from an outlet of the pneumatic nebulizer in a predetermined direction in a closed space; and
projecting UV-C radiation in the predetermined direction.

51. The method of claim 50, wherein the liquid peracetic acid is 2% w/w-15% w/w aqueous peracetic acid solution.

52. The method of claim 50, wherein the liquid peracetic acid is 5% w/w aqueous peracetic acid solution.

53. The method of claim 50, wherein the UV-C radiation has a wavelength of 100-280 nm.

54. The method of claim 50, wherein the UV-C radiation has a wavelength of about 254 nm.

55. The method of claim 50, wherein the aerosol of peracetic acid droplets includes a majority of droplets, by number, of size 4 μm or less.

56. The method of claim 50, further comprising:
introducing humidity into the closed space.

57. The method of claim 50, wherein compressed gas is used for the evaporating of the aerosol of peracetic acid droplets to peracetic acid vapour and the propelling of the peracetic acid vapour away from an outlet of the pneumatic nebulizer in the predetermined direction.

58. The method of claim 50, wherein a fan is used for the evaporating of the aerosol of peracetic acid droplets to peracetic acid vapour and the propelling of the peracetic acid vapour away from an outlet of the pneumatic nebulizer in the predetermined direction.
